(19) United States
(12) Patent Application Publication
(10) Pub. No.: US 2005/0169967 A1
(43) Pub. Date: Aug. 4, 2005

(54) Surgical material comprising water glass fibres

(76) Inventors: Thomas Gilchrist, Ayr (GB); David Healy, Ayr (GB)

Correspondence Address: Daniel A Monaco;Drinker Biddle & Reath, One Logan Square, 18th & Cherry Streets, Philadelphia, PA, 19103-6996, US

(21) Appl. No.: 10/513,241

(22) PCT Filed: May 2, 2003
(86) PCT No.: PCT/GB03/01918

(30) Foreign Application Priority Data
May 3, 2002 (GB) 0210517.9

(51) Int. Cl. A61F 2/00

(52) U.S. Cl. 424/426; 442/123

(57) ABSTRACT

There is provided a surgical material formed using water soluble glass fibres, optionally together with a binding material such as polycaprolactone. One or more layers of non-woven glass fibre material may be present. The material, which is usually in sheet form, can be folded around a defective area of tissue to promote healing, or may be used to prevent adhesion formation following surgery. A method of forming the surgical material is also described.

The present invention relates to a flexible biodegradable material which is particularly useful for tissue repair and tissue engineering.

Tissue damage can result from a variety of sources, particularly from trauma, disease or as the result of surgery. It is well known that following damage the healing of many tissues progresses slowly or, indeed, may not happen at all.

Healing of certain tissues (such as ligaments, tendons or internal organs) may be further hindered by the formation of adhesions between the damaged tissue and surrounding tissues. This is known to be a particular problem in tendon/ligament damage and also after surgery, for example heart surgery (when the heart can form adhesions with the back of the sternum) or abdominal surgery. Complications caused by adhesions include poor recovery, substantial morbidity and even catastrophic haemorrhage during reoperative surgery following adhesion of cardiac tissue (Nkere, U. U., ASIO Journal, 2000, Vol 46, pages 654-656). There is currently no reliable way to prevent formation of such adhesions.

Skin damage following severe burns or ulcers, such as diabetic foot ulcers, is notoriously difficult to heal. This is primarily because the dermis cells will not regenerate in the absence of a matrix on which to grow. Conventional skin grafts (autografts) generally result in scarring and necessitate creation of a further wound in the patient to obtain the graft. Use of non-autologous skin grafts brings the risk of rejection. Recently the development of tissue engineering and, in particular, artificial skin has presented advances in this area. Notable products are keratinocyte seeded Integra™, Dermagraft™, and Apligraft™ which contain neonatal cells in combination with matrices formed from bovine collagen or the soluble suture materials polylactic and polyglycolic acids. These artificial skins provide a matrix for dermis growth and the neonatal cells contained in them produce growth factors which promote healing. These prior art products are, however, unsatisfactory because of health concerns regarding bovine derived collagen and because the dissolution rates of suture materials cannot be tailored to specific needs. Mulder, G. T. (Journal of Wound Care, 1998, Vol 8, No 1, pages 21-23) discusses the shortcomings of autografts (including pain, infection, and delayed closure) and the advantages and problems associated with tissue engineering. Naughton, G. et al. (Artificial Organs 1997, Vol 21, No 11, pages 1203-1210) discusses clinical trials and successes of the Dermagraft™ product in the treatment of diabetic foot ulcers. Hollander, D. et al. (Journal of Wound Care, 1999, Vol 8, No 7, pages 351-355) discusses the success of treatment ulcers of different aetiologies using autologous keratinocytes cultured on benzylester hyaluronic acid membranes then applied as autologous grafts.

Healing of nerve fibres is also known to be particularly troublesome. Nerves regenerate following injury caused by trauma or disease through a biological process which, in the absence of outside assistance, is typically extremely slow and incomplete and frequently does not occur at all. Thus patients with nerve damage who do not receive expert surgery will frequently fail to regain the function of the damaged nerves. Early nerve repair is associated with a better outcome and delay should be resorted to only if life-threatening problems exist coincidentally (Glasby, M. A., et al. 1997, Journal of Hand Surgery, Vol 22B, No 4, pages 479-485; Galsby, M. A., et al. 1998, Journal of Hand Surgery, Vol 23B, pages 354-359; and Lawson, G. M. et al. 1995, Journal of Hand Surgery, Vol 20B, No 5, pages 663-670). Surgical methods of improving the healing of nerves are concerned with optimising the biological environment surrounding the damaged nerves and hence promoting the natural healing process. Over the past decade, interest has arisen in the use of neurotrophic factors as an adjunct to nerve repair. An early problem in the use of these substances was their supply, at an appropriate concentration, at the site of injury. Modern microsurgery techniques are currently used to achieve this but are costly, time consuming and require highly trained surgeons. An alternative which is now being explored is the use of tubes to surround damaged nerves and provide a favourable biological environment for nerve repair. In the case of non-biodegradable tubes this technique has achieved relatively little success, primarily because the tubes remain in place after the nerve fibre has regenerated and prevent subsequent maturation of the nerve, or necessitate a second operation to remove the tube. Silicone is the most extensively investigated non-biological conduit. However there have been problems with inflammation and compression of the nerve, in some cases requiring a second procedure to remove the conduit. Kiyotani, T. et al. (ASAIO Journal, 1995, Vol 41, pages 657-661) describes the use of biodegradable tubes made from collagen and polyglycolic acid in regenerating sciatic nerve damage in cats. Nerve regeneration of gaps of 25 mm was achieved.

WO-A-96/31160 (Giltech Limited) discloses a tubular device made from water soluble glass to promote healing of nerves, tendons or muscles which optionally contains a substance to promote healing. The devices described are inflexible and are not easily adaptable for different applications and may require considerable dexterity by the surgeon to implant correctly.

Additionally some difficulties were associated with the implantation of rigid glass tubes, and swelling was frequently produced at the site of implantation which caused discomfort and irritation for patients. Furthermore to provide for the entire range of injuries, a large number of differently sized tubes is necessary. The rigid tube may often not be an exact fit for the tendon, nerve or bone which it surrounds.

WO-A-00/47245 (Giltech Limited) discloses a rigid water soluble composite. The composite is formed from water soluble glass fibres set in a biodegradable polymer and is particularly useful for bone repair. The composite may be moulded into shapes as required by a particular application. The uses of this composite are, however, limited due to the need to pre-form the composite into the desired shape. The rigid nature of the composite precludes manipulation and reshaping of the composite once formed.

There thus remains a need for adaptable biodegradable and bio-compatible materials for use in adhesion prevention, tissue engineering and to promote healing.

The present invention provides a flexible biodegradable material comprising water soluble glass fibres and being suitable for implantation in a human or non-human animal body. The material of the invention is bio-compatible and will preferably promote or enhance healing of any damaged surrounding tissue.

The biodegradable material of the present invention is preferably in the form of a flexible sheet.

The flexible biodegradable material may comprise one or more coherent layers of water soluble glass fibres. Each of the layers preferably comprises a non-woven web of water soluble glass fibres.

The implantation of the flexible biodegradable material of the present invention does not require specialist equipment or microsurgery training and is thus ideal for use on the battle field or in the developing world where surgical expertise may be limited.

Furthermore, conduits formed from the material of the present invention support nerve regeneration over several centimetres and can therefore be used as an alternative to nerve grafting without the donor site morbidity involved with the latter process. The use of a conduit results in less damage to nerve ends as no sutures are required. Conduits formed from the material of the present invention can also be used to study the process of nerve growth and act as a reservoir for growth factors in the chemical enhancement of nerve regeneration.

The flexible biodegradable material of the present invention is easy to attach to nerve stumps and occupies minimal space in the wound cavity. As the material of the present invention may be cut to size it provides an exact fit around the tissue member.

As discussed above, tendon and ligament repair is confounded by adhesions of the tendon or ligament at the site of its repair to the surrounding tendon or ligament sheath. This invariably leads to poor healing of the tendon or ligament but can also lead to substantial morbidity or catastrophic haemorrhage. Instances of adhesion are commonly associated with the rigid tubes known in the prior art. Surprisingly it has been found that very little proliferation of connective tissue at the site of injury is observed when the flexible biodegradable material of the present invention is used to surround ligaments or tendons. The formation of adhesions is reduced.

The ideal conduits formed from the material of the present invention are non-biological, inert and dissolve over time so there is no permanent foreign body. Water soluble glass has the added advantage that it can be produced in a flexible glass fabric which can be adapted to different sizes of nerves.

Suitable water soluble glass fibres which may be used to form the flexible sheet are known in the art. These fibres, as is described later, may be selected to allow accurate tailoring of dissolution rate and/or the controlled release of selected ions.

Examples of compositions suitable for the production of water soluble glass fibres for the flexible biodegradable material include compositions comprising:
0 to 35 mole % of Na₂O
0 to 30 mole % of CaO
35 to 55 mole % P₂O₅
0 to 5 mole % of transition metal oxide
0 to 20 mole % MgO
0 to 10 mole % ZnO
0 to 10 mole % K₂O
0 to 8 mole % B₂O₃
0 to 10 mole % SO₃ and
0 to 5 mole % of NaF, Na₂PO₃F, 2Al₂O₃.B₂O₃, Na₂SO₄, FePO₄, MnHPO₄, Fe₂O₃ or Na₂B₄O₇. 10H₂O.

Generally compositions suitable for production of the flexible biodegradable material comprise:
15 to 25 mole % of Na₂O
10 to 15 mole % of CaO
45 to 50 mole % P₂O₅
0 to 5 mole % of transition metal oxide
0 to 5 mole % MgO
5 to 10 mole % ZnO
3 to 8 mole % B₂₀₃ and
0 to 3 mole % Fe₂O₃.

Examplary compositions include:

| | Mole % | | | | | | | |
|---|---|---|---|---|---|---|---|---|
| Composition | Na₂O | CaO | P₂O₅ | M₂O | MgO | ZnO | B₂O₃ | Fe₂O₃ |
| 1 | 25 | 13 | 48 | — | — | 8 | 5 | 1 |
| 2 | 20 | 13 | 49 | — | 4.25 | 8 | 5 | 0.75 |
| 3 | 23 | 12 | 48 | 3 | — | 8 | 5 | 1 |
| 4 | 18 | 12 | 49 | 3 | 4.25 | 8 | 5 | 0.75 |

(where M₂O is a suitable transition metal oxide or K₂O).

Whilst any suitable biocompatible water soluble glass may be used, phosphorous pentoxide (P₂O₅) is preferably used as the glass former.

Generally the mole percentage of phosphorous pentoxide in the glass composition is less than 85%, preferably less than 60% and especially between 30-60%.

Alkali metals, alkaline earth metals and lanthanoid oxides or carbonates are preferably used as glass modifiers.

Generally, the mole percentage of alkali metals, alkaline earth metals and lanthanoid oxides or carbonates is less than 60%, preferably between 40-60%.

Boron containing compounds (e.g. B₂O₃) are preferably used as glass additives.

Generally, the mole percentage of boron containing compounds is less than 15% or less, preferably less than 10%, and usually around 5% or less. Other compounds may also be added to the glass to modify its properties, for example SiO₂, Al₂O₃, SO₃ or transition metal compounds (e.g. first row transition metal compounds). Generally, the glass will release ionic species upon dissolution, the exact ionic species released depending upon the compounds added to the glass. Glasses which release aluminium ions, sulphate ions or fluorine ions may be desirable in some circumstances.

Typically the soluble glasses used in this invention comprise phosphorus pentoxide (P₂O₅) as the principal glass-former, together with any one or more glass-modifying non-toxic materials such as sodium oxide (Na₂O), potassium oxide (K₂O), magnesium oxide (MgO), zinc oxide (ZnO) and calcium oxide (CaO). The rate at which the glass dissolves in fluids is determined by the glass composition, generally by the ratio of glass-modifier to glass-former and by the relative proportions of the glass-modifiers in the glass. By suitable adjustment of the glass composition, the dissolution rates in water at 38° C. ranging from substantially zero (e.g. 0.002 mg/cm²/hr) to 25 mg/cm²/hour or more can be designed. However, the most desirable dissolution rate R of the glass is between 0.005 and 2.0 mg/cm²/hour.

The water-soluble glass is preferably a phosphate glass. Other metals may alternatively or additionally be present and mention may be made of Cu, Mg, Zn, Ce, Mn, Bi, Se, Cs. Preferred metals include Cu, Zn and Mg. The glass preferably enables controlled release of metal and other constituents in the glass and the content of these additives can vary in accordance with conditions of use and desired rates of release, the content of metal generally being up to 5 mole %. While we are following convention in describing the composition of the glass in terms of the mole % of oxides, of halides and of sulphate ions, this is not intended to imply that such chemical species are present in the glass nor that they are used for the batch for the preparation of the glass.

The optimum rate of release of metal ions into an aqueous environment may be selected by circumstances and particularly by the specific function of the released metal. The glass used in this invention provides a means of delivering metal ions to an aqueous medium at a rate which will maintain a concentration of metal ions in said aqueous medium of not less than 0.01 parts per million and not greater than 10 parts per million.

In some cases, the required rate of release may be such that all of the metal added to the system is released in a short period of hours or days and in other applications it may be that the total metal be released slowly at a substantially uniform rate over a period extending to months or even years. In particular cases there may be additional requirements, for example it may be desirable that no residue remains after the source of the metal ions is exhausted or, in other cases, where the metal is made available it will be desirable that any materials, other than the metal itself, which are simultaneously released should be physiologically harmless. In yet other cases, it may be necessary to ensure that the pH of the resulting solution does not fall outside defined limits. Generally, the mole percentage of these additives in the glass is less than 25%, preferably less than 10%.

In one embodiment of the present invention the flexible biodegradable material comprises one or more non-woven coherent layers of water soluble glass fibres. Optionally the layer(s) are needle-punched to form a non-woven felt. In general, in this embodiment, the flexible biodegradable material may consist substantially of water soluble glass. For example, the flexible biodegradable material may consist of 95% by weight or greater of water soluble glass.

In another embodiment of the present invention the flexible biodegradable material comprises one or more non-woven coherent layers of water soluble glass fibres wherein regions of the fibres are fused together. Fusion of the fibres may occur through any suitable means, for example by partial melting or sintering of the fibres or by partial dissolution of the fibres with water or any other suitable solvent, followed by solidification or evaporation respectively. In general, in this embodiment, the flexible biodegradable material may consist substantially of water soluble glass. For example, the flexible biodegradable material may consist of 95% by weight or greater of water soluble glass.

Optionally, a needle-punched felt of the first embodiment may undergo fusion by partial melting/sintering or by partial dissolution as described above.

In a further embodiment the present invention provides a flexible biodegradable composite material, comprising water soluble glass fibres and a bio-compatible binding material. The composite is suitable for implantation in a human or non-human animal body.

In one aspect of the above embodiment the bio-compatible binding material is coated onto the surface of the glass fibres. Alternatively the binding material may comprise a film fused to one or both sides of the layer of water soluble glass fibres. Films of binding material may also be sandwiched between two or more layers of the water soluble glass fibres.

Suitable bio-compatible binding materials include non-biodegradable polymers (such as nylon, polyester, polycarbonate, polypropylene, polyethylene, silicones, polyurethanes, PVC, polymethyl methacrylates and cyanoacrylates), biodegradable polymers (such as polymers of polycaprolactones, polyglycolic acid, polylactic acid, lactide/glycolide co-polymers) and natural materials (such as alginates, chitosans, starches, polysaccharides, collagen, skin, milk proteins, blood components including platelets or the like).

Preferably the bio-compatible binding material is a biodegradable polymer, particularly one of the biodegradable polymers listed above.

Preferably the bio-compatible binding material is polycaprolactone.

Preferably the amount of binding material in the composite material is less than 50% by weight, for example is less than 30% by weight.

The bio-compatible binding material may further comprise water soluble glass in powder form.

The level of permeability of the flexible biodegradable material of the present invention may be selected to permit a particular degree of movement of biological agents across the material.

Where the flexible biodegradable material is a non-woven felt and/or is formed from fused layer(s) of water soluble glass fibres, the level of permeability may be adjusted by increasing the number of layers (to decrease permeability) or decreasing the number of layers (to increase permeability). Additionally or alternatively, the permeability of the material may be adjusted through the needle felting process (the greater the density of needles, the lower the permeability and vice versa) and/or by adjusting the density of the fusion points (increased density of fusion equating to decreased permeability and vice versa). Where a composite material is under consideration, the binding material selected may affect permeability. Through adjustment of the permeability of the material, a substantially isolated biological environment could be achieved using an occlusive material to surround a tissue such as a nerve fibre or bone. Alternatively a diffusion permitting material could be used where isolation is not desirable, e.g. in poorly vascularised tissue. The use of a film of binding material is particularly suitable for controlling the permeability level of the composite material as the precise character of the film may be determined during manufacture. Preferably the flexible biodegradable material is sterilised, for example by gamma-irradiation. One particular advantage of the present invention is that the water soluble glass fibres are not degraded by this method of sterilisation.

In a further embodiment of the present invention the flexible biodegradable material may further comprise additives such as cytokines, cells or other biological agents. In this respect mention may be made of:
Nutritional agents, such as vitamins, oxygenators and free radical scavengers, and proteins;
Growth factors, (especially growth factors specific for the type of tissue concerned) such as platelet released and platelet derived growth factor, nerve growth factor, keratinocyte stimulation factors, insulin-like growth factors, ketanserin (a serotonomic blocking agent);
Living cells, for example keratinocytes or fibroblasts;
Enzymes, including streptokinase and streptodormase;
Elements such as zinc, selenium, cerium, copper, manganese, cobalt, boron, arsenic, chromium, gold, gallium;
Charcoal;
Desloughing and debriding agents such as hypochlorite and hydrogen peroxide;
Astringents including potassium permanganate; and/or
Anti-adhesiogenic substances—particularly triamcinolone.

In a further embodiment the flexible biodegradable material may function as a delivery device for pharmacologically active agents. This may be achieved, for example, by the controlled release of metal ions contained in the water soluble glass fibres. Alternatively or additionally where the flexible biodegradable material is a composite material comprising a binding material, the binding material may contain a pharmacologically active agent. For example water soluble glass powders may be present in the binding material. Alternatively other medicaments, exemplified by but not limited to those listed above, may be released by the flexible biodegradable material. These agents may be initially retained in the structure of the flexible biodegradable material and released as the material degrades in vivo.

In a further aspect, the present invention provides a method of treating an area of defective tissue in a patient, said method comprising using a flexible biodegradable material as described above to surround, cover or isolate said area of tissue. Optionally the material is attached to healthy or defective tissue by conventional means such as staples, sutures or biodegradable adhesive.

The tissue is suitably nerve, tendon, ligament, bone, skin, internal organ (for instance heart or intestine), dura matter, muscle, cartilage, blood, or lymph vessels and ducts.

In a further embodiment the present invention provides use of a flexible biodegradable material as described above in the treatment of an area of defective tissue, for example to protect said area of defective tissue, to promote healthy healing thereof or to prevent adhesion formation.

In a further embodiment the present invention provides the use of the flexible biodegradable material, as described above, in the manufacture of a surgical implant. In particular the implant may be useful for the treatment of tendon, nerve, skin and bone damage or to prevent adhesion.

In one embodiment the flexible biodegradable material is positioned between two internal tissue surfaces to prevent or reduce the formation of adhesions. This is particularly appropriate in treating tendon/ligament damage or after surgery (especially cardiac or abdominal surgery).

In another embodiment the flexible biodegradable material is formed into a tube around the area of damaged tissue (for example nerve, ligament, tendon or bone tissue). This may be achieved by simply wrapping or folding the material around the damaged tissue and then sticking, sewing or stapling the material into the desired conformation. In such applications creating an isolated biological environment for repair is often useful in addition to providing an element of structural support for directing tissue growth.

In a further embodiment the flexible biodegradable material may be used as dressing to cover an external area of tissue damage. This application is particularly appropriate for tissue damage caused by burns or diabetic ulcers, though other forms of dermal damage may also be treated. Here the material may act as a scaffold for adhesion and growth of dermal cells. In addition dermal cells can be provided on the material prior to application to a patient to further promote healing.

In a particular embodiment, the present invention provides a method of reducing adhesion formation following surgery in a patient. In this embodiment a sheet or pre-formed portion of the biodegradable flexible material is inserted into the patient during surgery and is located between the surfaces where cohesion formation is likely. Optionally the material may be fixed into place, for example using biodegradable adhesive, but this is not always necessary. The biodegradable material would be manufactured to degrade over the appropriate healing period, typically 1 to 3 months.

Likewise in an alternative embodiment the present invention provides a method of repairing a damaged tendon or nerve. Here the flexible material is simply wrapped around the damaged nerve or tendon and sealed in place by surgical glue. Where the nerve or tendon is severed, the two ends are located together and then held in place by wrapping and fixing the material as before.

In a further aspect the present invention provides a method of producing a flexible biodegradable material in sheet form, suitable for implantation into a patient's body, said method comprising:
a) providing one or more layer(s) of water soluble glass fibres; and
b) forming the fibres into a coherent layer by at least one of the following steps:
   i) fusing the fibres together by partial melting or dissolution; or
   ii) needle-punching the layer of fibres to form a non-woven felt; or
   iii) providing a binding material to adhere said layer to form a composite sheet.

In one embodiment the layer of water soluble glass fibres is formed by:
a) winding glass fibre(s) onto a drum;
b) cutting along the length of the drum and removing the substantially aligned glass fibres therefrom; and
c) pulling the glass fibres in a lateral direction to form a non-woven web.

In one embodiment the binding material may be adhered to the water soluble glass fibres by:
a) melting or dissolving the binding material in an appropriate solvent;
b) applying the binding material to the fibres by dipping, spraying or pouring to form a fibre/binding material composite; and
c) cooling, curing or drying the fibre/binding material composite.

In another embodiment the binding material is produced as a film and is then adhered to the water soluble glass layer by heat, solvent or adhesive.

Examples of uses and benefits of the flexible biodegradable material include:

Peripheral nerve repair: Where a peripheral nerve requires repair due to trauma or disease the flexible biodegradable material can be wrapped around the damaged area and fixed with adhesives or sutures. This system has advantages over existing peripheral nerve repair procedures in that it is very fast, requires less skill than a microsurgical repair and requires no sophisticated microsurgical equipment.

Tendon and ligament repair: Flexible biodegradable material fixed around recovering tendons and ligaments will prevent the formation of adhesions and subsequent damage to the bearing surfaces of the tendons.

Orthopaedics: The flexible biodegradable material can be used to enclose fracture sites and defects and contain bone fragments, chips or synthetic bone materials as well as other growth/repair factors at the implant site. The material can also be used, as a heavier sheet, as a scaffold for low load bone repairs such as orbital repair.

Skin equivalents: With the appropriate combination of fibre(s) and binding material(s), skin equivalent systems may be used as support and implantable delivery substrates for skin repair. These materials can be used to grow various cell types on prior to transfer to the patient, or used directly in vivo.

Woundcare: The flexible biodegradable material can be used to deliver blood platelets and growth factors to wounds to encourage rapid recovery.

Dura mater equivalent: The flexible biodegradable material may be used as an equivalent to the dura mater where it has been damaged or removed by trauma or surgical intervention.

Cardio thoracic: Cardio thoracic surgery would benefit from use of the flexible biodegradable material to assist wound closure without encouraging adhesion formation.

Slings: The flexible biodegradable material may prove to be an easy to use sling for incontinence and hernia repair procedures.

Hole patches: The flexible biodegradable material sheets may be used for the repair of holes, such as stab or gunshot wounds, in the body created by trauma (heart, lungs, digestive tract, cut or torn blood vessels, etc).

The flexibility of the material allows it to be used in repairs where mobility is needed. In the convenient sheet form, the material can be manipulated to conform to any shape and can be thermoformed to produce shapes of the desired size and contour at the site of use. Since the flexible material dissolves completely it will not cause fibrous tissue occlusion of the repaired nerve (as may occur with non-biodegradable materials). The flexible biodegradable material can be used around tissues which have not been severed (tendons, ligaments, crush injuries) where the local environment requires temporary control.

Where appropriate, the surgeon may secure a damaged area with more than one layer of flexible biodegradable material.

Embodiments of the invention will be described in the following non-limiting examples with reference to the accompanying drawings in which:

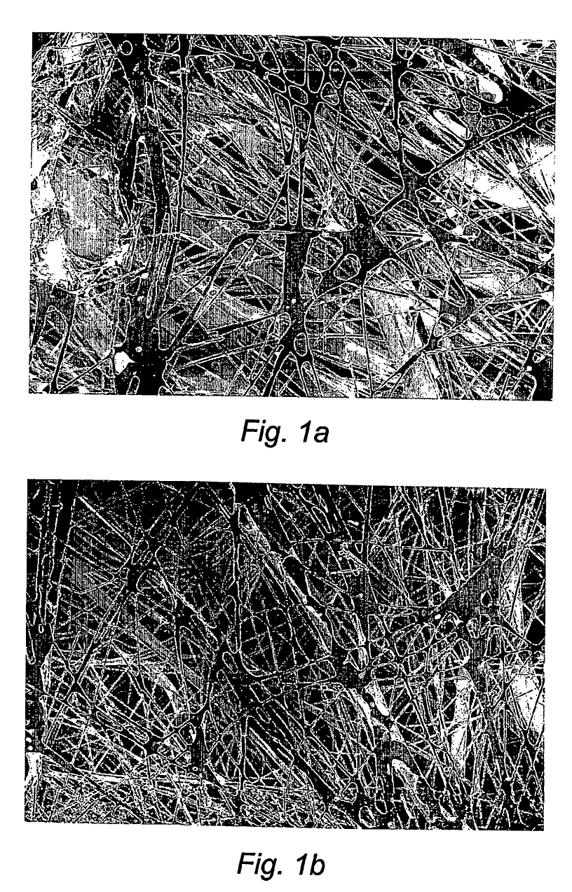

FIGS. 1a and 1b show Scanning Electron Microscopy (SEM) images (×100) of both sterile (a) and non-sterile (b) flexible biodegradable material manufactured in accordance with the present invention.

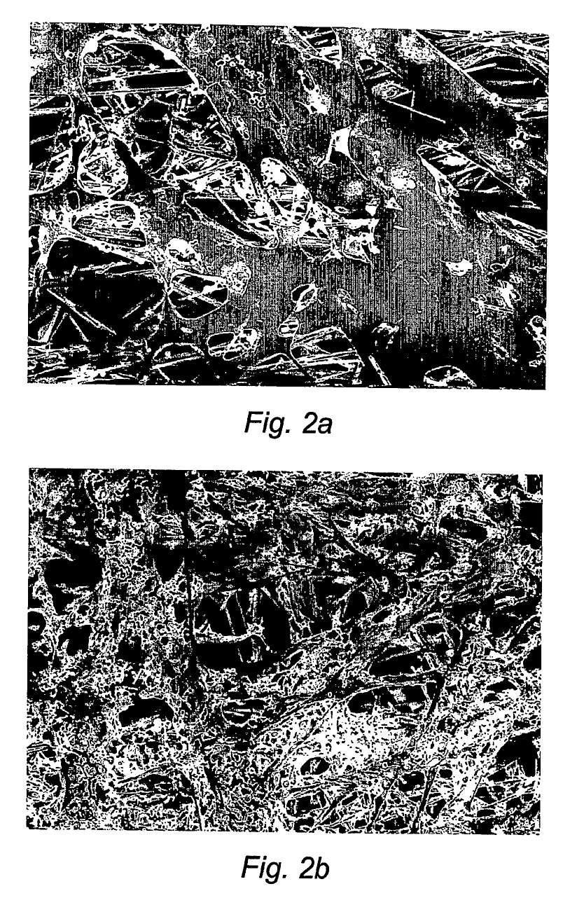

FIGS. 2a and 2b show SEM images (×100) of both sides of a flexible biodegradable material according to the invention following 48 hours incubation with L929 fibroblasts.

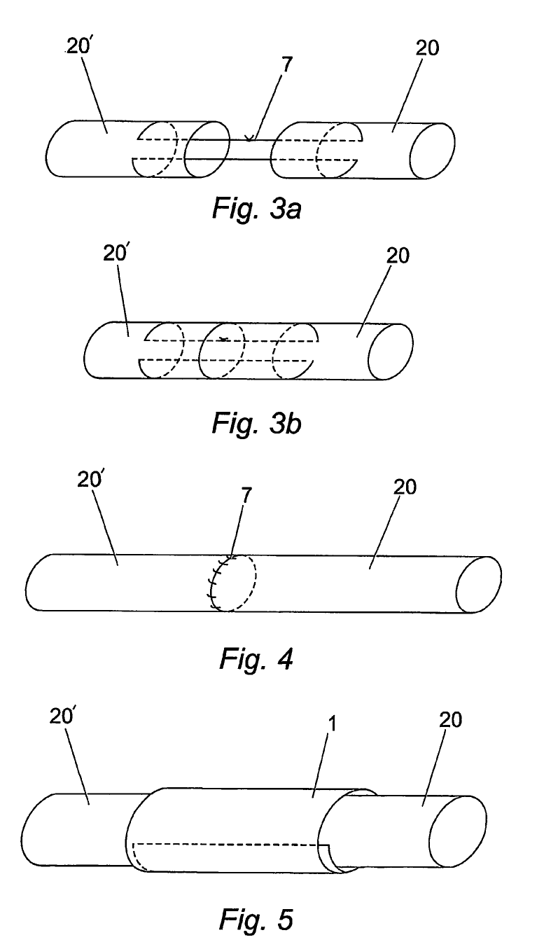

FIG. 3 shows the modified Kesseler repair of a tendon.

FIG. 4 shows epitenon repair of a tendon.

FIG. 5 shows the flexible biodegradable material wrapped around a tendon following repair.

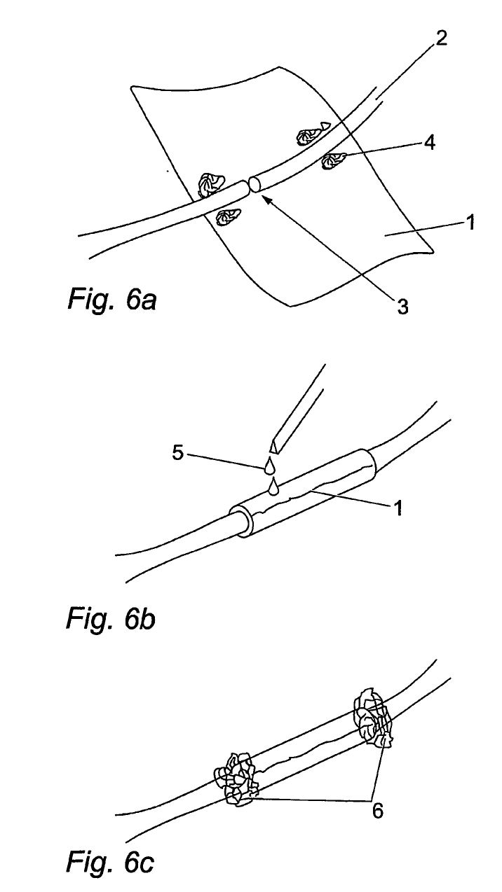

FIG. 6 shows use of the flexible biodegradable material in nerve repair.

### EXAMPLE 1

### Method of Forming a Glass Fibre

The glass-forming composition is initially heated to a melting temperature of 500°-1200° C., preferably 750°-1050° C. The temperature is then slowly lowered to the working temperature at which fibre formation occurs. Generally, the working temperature of the glass will be at least 200° C. lower than the temperature at which the glass is initially heated. Suitable working temperatures may fail within the following ranges 400°-500° C., 500°-900° C. (preferably 550°-700° C., more preferably 550°-650° C., especially 600°-650° C.) and 800°-1000° C. The working temperature selected will depend upon the glass composition, but an approximate indication of a suitable working temperature can be established as hereinafter described. Depending upon the glass composition used, the working temperature may be a range of suitable temperatures. The range of working temperatures may be narrow, for example of only 10° C., so that fibre formation may occur only between the temperature of N° C. to (N+10)° C. Other glass compositions may have a wider temperature range for the working temperature in which glass formation is possible.

Alternatively, the working temperature of the glass may be defined as 50-300° C. above the Tg of the glass.

In order to obtain an approximate indication of the working temperature for any particular glass composition, the glass composition should be slowly heated to its melting point. As soon as the glass is molten, frequent attempts to pull the composition upwardly to form a fibre should be made, with the temperature of the composition being very gradually increased between attempts. The temperature range of the composition during which fibre formation is possible should be noted and used as a preliminary working temperature in the process of the invention.

It will be clear to those skilled in the art that the pulling speed at which the fibre is drawn off can affect the choice of working temperature and the diameter of the fibre required. Where a fibre of relatively large diameter is required, the fibre tends to be pulled more slowly and the working temperature may need to be decreased slightly. Where a fibre of relatively small diameter is required (e.g. a glass wool), the fibres may be drawn at the much higher pulling speed and the working temperature may need to be increased (thus lowering the viscosity of the composition to accommodate the increased pulling speed). Selection of the exact working temperature in respect of any particular fibre size and composition will be a simple matter of routine evaluation of optimal process conditions.

With reference to the “working temperature” of the glass, the skilled person will appreciate that the furnace temperature may differ considerably from the temperature of the glass itself and indeed there may be a significant temperature gradient in the glass. Ideally the “working temperature” will be the temperature of the glass as fibre formation (i.e. pulling) takes place. In many compositions however, it may not be practical to measure the temperature at the surface of the glass where pulling occurs by insertion of a temperature probe as the introduction of the probe may precipitate crystallisation of the glass. One alternative is to place a temperature probe into the bushing and to monitor the bushing temperature which will be a good indicator of the glass temperature at the moment of fibre formation. Alternatively an Infra Red pyrometer may be focused onto the appropriate area of the glass and used to monitor the temperature.

The glass to be formed into fibres will generally be heated until molten, optionally clarified, and then cooled slowly and controllably until the appropriate working temperature is reached and fibre formation can commence. The initial heating of the glass above its melting point and the subsequent fibre formation may be carried out in a single vessel or, alternatively, the molten glass may be transferred to a vessel designed specifically for fibre formation. One way of holding the molten glass in a vessel having a bushing within its lower surface until the temperature drops to the required working temperature is to coat or fill the holes of the bushing with a material that gradually melts over the period of time taken for the glass to reach the temperature required.

The most important aspect of the method is the manner in which the working temperature is reached.

We have found that the molten glass, which may preferably be heated significantly above its melting point, should be allowed to cool in a highly controlled manner, the temperature being only gradually reduced until the working temperature is reached. A stirrer may be present to ensure that the temperature of the whole of the molten glass is kept as uniform as possible.

The glass is cooled to a temperature at which the glass will not crystallise for at least the period of time needed to convert the melt to fibre. This temperature is termed herein as a “holding temperature”. The rate of cooling from this holding temperature is determined by the rate at which the melt is consumed at the bushing and the difference in temperature between the bushing temperature (the working temperature) and the melt holding temperature.

Due to low viscosity and narrow temperature band for many of these compositions, control of the balance between melt temperature, bushing temperature and glass throughput rate is critical.

### EXAMPLE 2

### Producing a Flexible Biodegradable Material

Glass fibres of desired composition are formed as described above in Example 1 using a multi-hole bushing, the fibres being wound onto a drum at high speed during production.

The following table shows water soluble glass compositions which are particularly suitable for producing fibres for producing a flexible biodegradable material:

| | Mole % | | | | | | | |
|---|---|---|---|---|---|---|---|---|
| Composition | Na₂O | CaO | P₂O₅ | M₂O | MgO | ZnO | B₂O₃ | Fe₂O₃ |
| 1 | 25 | 13 | 48 | — | — | 8 | 5 | 1 |
| 2 | 20 | 13 | 49 | — | 4.25 | 8 | 5 | 0.75 |
| 3 | 23 | 12 | 48 | 3 | — | 8 | 5 | 1 |
| 4 | 18 | 12 | 49 | 3 | 4.25 | 8 | 5 | 0.75 |

(where M₂O is a transition metal oxide, or K₂O).

It should be understood, however, that these examples are non-limiting and other water soluble glass compositions may be suitable.

The windings of collected fibres are then cut perpendicular to their direction, i.e. the cut is made longitudinally along the surface of the drum, and the windings removed from the drum as a bundle of fibres (the uniform length of the fibres being the same as the drum circumference). At this point, all the fibres are substantially aligned in the same direction.

The bundle of fibres is then laid flat on a clean surface and one of the non-cut edges is gently teased sideways away from the bundle. As the edge is pulled out the fibres expand to form a non-woven web; the arrangement of the fibres being intrinsically interlinked and the web resembles the wires in a chain-link fence. This intertwining of wound fibres and the consequent nature of expansion upon pulling is a known property of conventional glass fibres.

Expansion is continued by pulling until the fibres of the web are well separated and a suitable amount of fibre material has been obtained. The weight and texture of the web are determined by the initial fibre properties, the degree of expansion and the thickness of the bundle from which the web is drawn. Several layers of the expanded web may be overlapped to obtain a layer of glass fibres of the desired thickness. This may conveniently be achieved by rolling the expanded web onto a further drum, the number of complete revolutions of the drum corresponding to the number of layers required. The fibres are then cut and the layer removed in sheet form in a manner similar to the earlier technique.

At this point the fibre layer could be heat bonded, partially dissolved or needle-punched in order to form a coherent material.

To form a composite material the layer is then conveniently placed on a releasable backing material (for example siliconised sheeting) and a binding material applied; for example, polycaprolactone 650 dissolved in chloroform (70 g/dm³) may simply be poured onto the layer. Releasable backing material is then put on top of the layer and the sandwiched material pressed flat. Once the chloroform has evaporated the composite material is peeled off from the releasable backing material. FIGS. 1a and 1b show SEM images of a composite material made according to the present invention. The material in FIG. 1a has been sterilised by exposure to γ-irradiation whereas the example shown in FIG. 1b has not; the structures appear substantially identical showing that γ-irradiation has not affected the fibre structure of the material.

Alternatively a pre-formed film of binding material could be positioned on one surface of the sheet of glass fibres and bound to the sheet by heating, applying a solvent or biodegradable adhesive.

The level of permeability of the composite material may be controlled by the nature of the binding material. For example, a perforated film or low amount of binding material results in an open structure that would allow the free passage of fluids, gasses and small particulates through the flexible composite material. Alternatively, use of an intact film or a large amount of binding material would render the flexible composite material occlusive, therefore limiting the passage of fluids and gases through the flexible composite material.

It will be clear to a person skilled in the art that a plurality of layers of glass fibre sheet and/or binding material film could be built up to produce a laminar material of desired properties. In addition layers of other materials such as alginates could be incorporated.

It will also be clear that a plurality of fibres of different properties could be employed to produce a composite material of desirable properties, e.g. combining a strong fibre with an antimicrobial fibre.

Conveniently, where a thermoplastic binding material (such as polycaprolactone) is used, the composite material may be shaped and moulded by manipulation in combination with heating, for example with a hairdryer.

The composite material can be supplied in sheet or roll form or can be pre-formed into various three dimensional shapes.

Examples 3 to 19 give alternative glass compositions suitable for fibre formation and thus for the production of the composite material using the methodology of Example 2.

### EXAMPLE 3

| | | |
|---|---|---|
| Na₂O | 31.19 | mole % |
| K₂O | 9.63 | mole % |
| M₂O | 2.9 | mole % |
| B₂O₃ | 2.74 | mole % |
| NaF | 0.66 | mole % |
| P₂O₅ | 52.88 | mole % |

(Where M₂O is a suitable transition metal oxide or potassium oxide).

Furnace at 710° C.-800° C.
Bushing at 450° C.-460° C.
4.5 mm bushing holes.
50 km per hour pull rate.
Good fibres.
Solution rate 1.68 not annealed 2.28 annealed.

### EXAMPLE 4

| | | |
|---|---|---|
| Na₂O | 32 | mole % |
| K₂O | 10 | mole % |
| M₂O | 3 | mole % |
| P₂O₅ | 55 | mole % |

(Where M₂O is a suitable transition metal oxide or potassium oxide).

Furnace at 850° C.
Bushing at 530° C.
5 mm bushing holes.
55 kmph.
Good strong fibres.

### EXAMPLE 5

| | | |
|---|---|---|
| Na₂O | 32 | mole % |
| K₂O | 10 | mole % |
| MgO | 4 | mole % |
| B₂O₃ | 5 | mole % |
| M₂O | 3 | mole % |
| P₂O₅ | 46 | mole % |

(Where M₂O is a suitable transition metal oxide or potassium oxide).

Furnace temperature 650° C.-730° C.
Bushing temperature 410° C.-420° C.
Bushing 5.5 mm diameter.
Speed up to 100 kmph.
Solution rate 0.7 annealed 1.0 non annealed (mg.cm⁻³.hr⁻¹)
Very good strong reliable fibre. Very stable.

Example 5 can be modified by replacing the MgO with ZnO.

### EXAMPLE 6

| | | |
|---|---|---|
| Na₂O | 36.68 | mole % |
| K₂O | 8.63 | mole % |
| P₂O₅ | 45.09 | mole % |
| B₂O₃ | 5.29 | mole % |
| M₂O | 2.59 | mole % |
| (CaO | 1.73 | mole % to attenuate solution rate) |

(Where M₂O is a suitable transition metal oxide or potassium oxide).

Furnace temperature 550° C.
Bushing 62×5.0 mm holes.
Bushing temperature 400° C.
Speed 80 kmph.
Very good fibres.

Solution rate 3.11 annealed, 3.8 non annealed (mg.cm⁻².hr⁻¹)

The fibres show excellent tensile strength, flexibility and shock resistance.

The fibres are especially suitable for rapidly biodegradable applications.

### EXAMPLE 7

| | | |
|---|---|---|
| Na₂O | 31.05 | mole % |
| CaO | 16.00 | mole % |
| M₂O | 3.88 | mole % |
| P₂O₅ | 46.08 | mole % |
| Na₂PO₃F | 0.97 | mole % |
| 2Al₂O₃.B₂O₃ | 2.00 | mole % |

(Where M₂O is a suitable transition metal oxide or potassium oxide).

100 grams of the sample was heated to 900° C. before being cooled and pulled at 650° C., at 25 km/hr. Overall the fibre was good; one sample was 10 km in length and 11 grams in weight, although there was some crystallisation at the pulling temperature.

### EXAMPLE 8

| | | |
|---|---|---|
| Na₂O | 29.51 | mole % |
| CaO | 15.21 | mole % |
| M₂O | 3.68 | mole % |
| P₂O₅ | 43.80 | mole % |
| 2Al₂O₃.B₂O₃ | 1.90 | mole % |
| Na₂PO₃F | 1.90 | mole % |
| Na₂B₄O₇.10H₂O | 1.00 | mole % |
| Na₂PO₄ | 3.00 | mole % |

(Where M₂O is a suitable transition metal oxide or potassium oxide).

74 grams of the sample was heated to 1000° C. before being cooled and pulled at 635° C. at 25 km/hr. The fibre produced was ultrafine; one sample was 18 km in length and 59 grams in weight. The sample was sprayed with WD40 to prevent water absorption and to aid lubricity. There was some debris at the bottom of the crucible, but this was found to be just iron deposits from the brushing rod.

### EXAMPLE 9

| | | |
|---|---|---|
| Na₂O | 34.20 | mole % |
| CaO | 16.15 | mole % |
| P₂O₅ | 44.65 | mole % |
| Na₂SO₄ | 5.00 | mole % |

200 grams of the sample was heated to 1050° C. before being cooled and pulled at 635° C. at 25 km/hr. The fibre was good although there was some crystallisation at the pulling temperature.

### EXAMPLE 10

| | | |
|---|---|---|
| Na₂O | 32.40 | mole % |
| CaO | 15.30 | mole % |
| P₂O₅ | 42.30 | mole % |
| 2Al₂O₃.B₂O₃ | 3.00 | mole % |
| Na₂PO₃F | 1.00 | mole % |
| Na₂SO₄ | 6.00 | mole % |

117 grams of the sample was heated to 950° C. before being cooled and pulled at 635° C., at 40 km/hr. The fibre produced was good and there were no crystallisation problems even though the surface temperature of the fibre dropped to 510° C. in the pulling process.

### EXAMPLE 11

| | | |
|---|---|---|
| Na₂O | 31.71 | mole % |
| CaO | 14.73 | mole % |
| P₂O₅ | 36.33 | mole % |
| B₂O₃ | 4.78 | mole % |
| SO₃ | 9.40 | mole % |
| Na₂PO₃F | 3.00 | mole % |

99 grams of the sample was heated to 800° C. before being cooled to 650° C. and pulled at 40 km/hr. The fibre produced was very fine but difficult to pull and quite fragile at speed.

### EXAMPLE 12

| | | |
|---|---|---|
| Na₂O | 30.77 | mole % |
| CaO | 14.28 | mole % |
| P₂O₅ | 35.28 | mole % |
| B₂O₃ | 4.64 | mole % |
| SO₃ | 9.12 | mole % |
| FePO₄ | 2.41 | mole % |
| Na₂PO₃F | 0.20 | mole % |
| MnHPO₄ | 2.06 | mole % |

200 grams of the sample was heated to 850° C. before being cooled to 545° C. and pulled at 40 km/hr. The fibre produced was strong and thin; there was not a problem of crystallisation, in fact the glass can be stored at 550° C. for 72 hours without the onset of crystallisation.

### EXAMPLE 13

Below is an example of a wool formulation and running conditions to illustrate the differences with the monofilament examples given above. A typical wool formulation is:

| | | |
|---|---|---|
| Na₂O | 26.31 | mole % |
| CaO | 17.78 | mole % |
| P₂O₅ | 47.04 | mole % |
| B₂O₃ | 5.94 | mole % |
| MnO | 1.55 | mole % |
| Fe₂O₃ | 0.97 | mole % |
| NaF | 0.41 | mole % |

Solution rate, non annealed=0.0278 mg.cm⁻² hr⁻¹
Melted and refined at 1000° C.
Cooled and held at 725° C.
Bushing temperature maintained at 365° C.

Thick fibres approx 1.2 mm diameter drawn through pinch rollers at 2.5 M.mm⁻¹ from a bushing with 6×6.5 mm diameter holes. Fibres jet attenuated to produce a fine wool 5-15 μm diameter. The wool was sprayed with silicone oil finish during the attenuation process and collected on a stainless steel mesh conveyor. Typically, attenuated wools will have diameters of 5 to 20 μm. Monofilament fibres will mostly be 20 to 50 μm diameter.

### EXAMPLE 14

| | | |
|---|---|---|
| Na₂O | 32 | mole % |
| K₂O | 10 | mole % |
| M₂O | 3 | mole % |
| P₂O₅ | 55 | mole % |

(Where M₂O is a suitable transition metal oxide or potassium oxide).

Furnace at 850° C.
Bushing at 530° C.
5 mm bushing holes.
55 kmph.
Good strong fibres.

### EXAMPLE 15

| | | |
|---|---|---|
| Na₂O | 32 | mole % |
| K₂O | 10 | mole % |
| MgO | 4 | mole % |
| B₂O₃ | 5 | mole % |
| M₂O | 3 | mole % |
| P₂O₅ | 46 | mole % |

(Where M₂O is a suitable transition metal oxide or potassium oxide).

Furnace temperature 650° C.-730° C.
Bushing temperature 410° C.-420° C.
Bushing 5.5 mm diameter.
Speed up to 100 kmph.
Solution rate 0.7 annealed 1.0 non annealed (mg.cm⁻³.hr⁻¹)
Very good strong reliable fibre. Very stable.

### EXAMPLE 16

| | | | |
|---|---|---|---|
| (K₂O | 5 | mole %) | Trace to alter |
| | | | dissolution rate |
| CaO | 25 | mole % | |
| MgO | 20 | mole % | |
| P₂O₅ | 50 | mole % | |

Furnace 1000° C.
Bushing 5.5 mm.
Bushing temperature 560° C.-620° C.
Speed up to 70 kmph.
Solution rate TBA.
Very strong fibre.

### EXAMPLE 17

| | | |
|---|---|---|
| CaO | 28.5 | mole % |
| MgO | 18.5 | mole % |
| M₂O | 3 | mole % |
| P₂O₅ | 50 | mole % |

(Where M₂O is a suitable transition metal oxide or potassium oxide).

Furnace temperature 1050° C.-1150° C.
Bushing 4×5.5 mm.
Bushing temperature 700° C.
Speed 50 kmph.
Solution rate TBA.
Very good, strong fibre.

### EXAMPLE 18

| | |
|---|---|
| CaO | 30 mole % |
| MgO | 20 mole % |
| P₂O₅ | 50 mole % |

The fibres show excellent tensile strength, flexibility and shock resistance. These fibres are suitable for applications requiring slower release and greater tensile strength plus biodegradability. The fibres are suitable for orthopaedic implants and tissue engineering applications.

### EXAMPLE 19

| | |
|---|---|
| CaO | 28 mole % |
| MgO | 20 mole % |
| ZnO | 10 mole % |
| P₂O₅ | 45 mole % |

The fibres show excellent tensile strength, flexibility and shock resistance. These fibres are suitable for applications requiring slower release and greater tensile strength plus biodegradability.

The fibres are suitable for orthopaedic implants and tissue engineering applications.

### EXAMPLE 20

### Prevention of Adhesion Formation Following Flexor Tendon Surgery in Sheep

This example demonstrates that the flexible composite material reduces adhesion formation and/or improves healing following tenotemy in sheep. The tendon to be severed is the pars superficialis of the flexor digitorum superficialis (FPS(PS)), the tendon in the ovine model being of comparable size to tendons of the human hand. This protocol would clearly also be applicable to a non-composite material such as a needle felt or heat fused material.

The current “gold-standard” procedure for tendon repair in clinical practice is that of modified Kessler core suture (see FIG. 3) reinforced by the addition of a circumferential epitenon suture (see FIG. 4). In FIG. 3 the two ends 20, 20′ of the severed tendon are pulled into close proximity by the suture 7. FIG. 3a shows the route of the suture and FIG. 3b shows the tendon ends 20, 20′ once pulled together by the suture 7. In FIG. 4, the modified epitenon repair is shown. Here, the severed ends of the tendon, 20, 20′ are held together by stitching using a suture 7.

However some controversy still exists about the addition of the epitenon suture (see FIG. 4) as, although it adds greatly to overall strength of the repaired tendon and “tucks in” the raw tendon ends, some believe it exacerbates the problem of adhesion formation postoperatively. Thus both forms of repair were evaluated in this study.

In the comparitive groups, repair of tenotomy is carried out by the modified Kesseler technique (FIG. 3) and additionally, in some animals, epitenon repair (FIG. 4). In the test groups composite material according to the invention was wrapped around the repair site (see FIG. 5). In FIG. 5 the spirally wrapped composite material 1 is shown in palce around the severed ends of the tendon 20, 20 and spanning across the site of repair. The composite material 1 is held in place by tissue glue or suture (not shown).

The following experimental groups were devised:
Group 1—Control.
Group 2—Tenotomy+modified Kessler repair.
Group 3—Tenotomy+modified Kessler repair+repair of epitenon.
Group 4—Tenotomy+modified Kessler repair+flexible composite material.
Group 5—Tenotomy+modified Kessler repair+repair of epitenon+flexible composite material.

Two cohorts of 12 animals were studied for each group. One cohort for each group was assessed at six weeks after surgery, the other cohort for each group was assessed six months after surgery. After six months the healing process would be expected to be complete. After six weeks the healing process would not be expected to be complete but assessment at this stage in the healing process allows investigation of the presence of early scar tissue. It is usual that early brisk tissue reaction produces a mass of connective tissue and the production of such tissue was compared with the permanent scarring resulting from adhesion formation.

Each operation was performed under general anaesthesia, with the full spectrum of non-invasive monitoring and with strict aseptic technique. The anatomical findings were observed to be consistent among individual animals. The techniques of creating the tendon division and of primary surgical repair were consistently reproducible. No unforeseen difficulties were encountered.

Surgical Technique:

All operations were carried out under sterile conditions using throughout techniques established in human surgery.

The tendon was approached through an incision beginning over the carpo-metacarpal joint and extended distally over the metacarpal bone. The tendon and muscle are invested by a fibrous sheath which is opened longitudinally to expose the tendon. Relieved of its sheath the tendon falls naturally into its two slips, the larger pars superficialis and the smaller pars profunda. The two slips derive from separate muscle bellies and run separately for most of their lengths before reuniting just proximal to their combined insertion into the middle phalanx. The pars profunda was left intact and the pars superficialis was severed at least 2 cm proximal to its junction with the pars profunda.

Tendons were repaired using the established modified Kessler technique which is an interwoven “core” suture designed to give maximum strength in the axis of the pull with minimal exposure of adhesiogenic suture material on the surface of the tendon (see FIG. 3). In selected groups this is supplemented by repair of the epitenon (see FIG. 4). Epitenon repair serves to improve strength of the repair, but may also cause an increase in the number of adhesions hence limiting movement.

After repair of the tendon, certain groups of sheep have the composite material according to the invention wrapped around the repair site (FIG. 5).

The composite material used water soluble glass fibres formed from the following composition:

Mole %

| Na₂O | CaO | P₂O₅ | MgO | ZnO | B₂O₃ | Fe₂O₃ |
|---|---|---|---|---|---|---|
| 25 | 13 | 48 | — | 8 | 5 | 1 |

in accordance with Example 2; the binding material was polycaprolactone.

The overlapping edges of the composite material were fixed together by polymer glue, although other suitable means such as sutures or “spot welding” with a cauterising tool may be appropriate. The composite material wrap was fastened in position on the tendon by a tissue glue (such as Tisseel™ glue).

Closure of the wound was by layers using conventional techniques and absorbable sutures throughout.

The animals were then allowed to recuperate for the specified time period (6 weeks or 6 months, depending on the experimental group).

After the specified time period had elapsed for each group a number of in vivo and in vitro tests were performed. Within groups-variation is reduced by expressing all measured variables as a fraction of that obtained from the corresponding site on the unoperated (left) side of each animal.

For all twelve animals in each group two in vivo (physiological) procedures were carried out. First tendon blood flow was measured by laser doppler flowmetry at two sites simultaneously; probe one (P1) sited proximal to the repair site and probe two (P2) distal to the repair site. This allowed assessment of blood flow around the area of repair.

Next the FDS(PS) tendon was divided at its distal end and attached to a displacement transducer. The FDS(PS) muscle was then triggered to contract by use of a transcutaneous nerve stimulator. The objective of this second procedure was to determine functional characteristics of the tendon's performance in situ after healing has occurred.

The FDS(PS) tendon was then harvested and in vitro (either mechanical or morphological) observations were carried out on groups of 6 animals. The mechanical analysis involves measuring the strength of the tendon using standard engineering methods, placing the specimen in a tensile testing machine (Instron). As this clearly results in destruction of the specimen, the remaining six specimens in each group were used for morphological analysis. This involves tissue processing of the sites of repair to allow histological sections to be prepared, stained and examined under microscopy for general histological appearance and calculation of percentage composition.

Results

Firstly with regard to doppler flux levels, in both time cohorts, Groups 2 and 3 (where repairs were performed without the flexible biodegradable material) showed markedly higher flux levels at the proximal (P1) probe compared to those of Groups 4 and 5. The repairs performed using the flexible biodegradable material (Groups 4 and 5) show P1 flux levels approximately equivalent to those of the normal, un-operated left side. This indicates a less florid vascular/adhesive response in the cases where the flexible biodegradable material was used. In a number of cases this can also be seen on gross inspection of the tendon, with large amounts of thickened vascular tissue surrounding the proximal area of tendon and repair site. These results show that use of the flexible biodegradable material allows tendons to heal whilst reducing the occurrence of adhesions.

The second important finding relates to the resultant ultimate tensile strength of the repaired tendons when tested in the Instron machine. In the six month groups, all repaired tendons, right FDS(PS), demonstrate a breaking strength equal to or greater than that of their own contralateral control tendon, left FDS(PS) (which was not operated on).

This suggests that the incorporation of the flexible biodegradable material encourages good healing of the tissue and does not inhibit or weaken the process of tendon healing.

In summary, the experiments described above indicated that;
there is not incompatibility between the implanted biodegradable material and the tissues involved in the healing process.
After repair with biodegradable material the recovered strength of the tendon is equal to or greater than that of their normal (opposite limb) controls. This is as good as might be expected for any sort of repair.
Blood flow studies around the tendons repaired with biodegradable material show a less florid response that where no material has been used. This suggests that less scar tissue is being formed.

### EXAMPLE 21

### Peripheral Nerve Repair Using Biodegradable Glass Fabric

This study is being carried out on sheep to demonstrate the potential of the composite material to promote peripheral nerve repair. The study comprised three experimental groups and one control group; all groups contain six sheep.

The surgical method involves neurotomy (complete severing of the nerve fibre) of the medial and facial nerve in the three experimental groups. Spontaneous recovery is never observed following neurotomy.

In two of the experimental groups the repair procedure shown in FIG. 6a-c is carried out, and composite material 1 is placed under the intact nerve prior to neurotomy. This is done for simplicity but the composite material could be placed in position after cutting. The nerve is then cut. As shown in FIG. 6a, the composite material 1 is in position under the nerve 2 which has been cut. The site of neurotomy is shown at 3. The composite material 1 is fastened in place on the nerve 2 by Tisseel™ glue 3 and FIG. 6a shows portions of such tissue glue 4 at each side of the nerve 2 on both edges of material 1. The composite material 1 is then wrapped around the nerve 2, the overlapping regions of material being bonded together by tissue or polymer glue 5 (see FIG. 6b) to hold the material 1 in a spirally wrapped conformation around the nerve. The exposed edges wrapped material 1 are then sealed by tissue or polymer glue 6 as shown in FIG. 6c. The manipulation and fastening of the composite material is a relatively simple procedure requiring considerably less skill than conventional micro-surgical nerve repair. A similar technique can be used for tendon repair. The composite material used in this protocol is the same as that in Example 20.

In the third experimental group the nerve is repaired by conventional end-to-end repair. The wounds are then closed and the animals allowed to recover for 6 months. Repair of the nerve fibre is examined by single-fibre electromyography, nerve conduction studies, target muscle isometric twitch, tetanic tensions and morphometric analysis. The results were subjected to statistical analysis.

There was no statistical significance between the groups in which the material of the present invention was used, and the group in which nerve is repaired by conventional end-to-end repair for any of the variables measured.

In this series of experiments, repair of the facial nerve in the sheep model using biodegradable composite material according to the present invention produced a level of recovery of function equal to that found after conventional epineurial repair. Since the latter method is widely used in clinical practice this suggests a surgical role for the composite material for repair of the facial nerve which obviates the need for microsurgical techniques.

### EXAMPLE 22

### Anti-Adhesiogenic Properties of the Flexible Biodegradable Material

In vitro direct contact tests were carried out with L929 fibroblast and neuroblastoma cell lines to examine the effects of the flexible composite material on cell proliferation and adhesion. Small pieces (approx 1 cm³) of flexible composite material were put into wells of 6-well plates with freshly subcultured fibroblasts or neuroblastoma cells. 0.1-0.2 cm³ of the freshly suspended cells at a concentration of 0.1-0.2×10⁶ cells/ml or 0.6-0.7×10⁶ cells/ml were added. The samples were prepared in duplicate, with duplicate control wells containing the fibroblast or neuroblastoma cells only (ie. no composite material). The water soluble glass fibres used in the manufacture of the composite material for the present protocol were of the composition:

Mole %

| Na₂O | CaO | P₂O₅ | ZnO | B₂O₃ | Fe₂O₃ |
|---|---|---|---|---|---|
| 25 | 13 | 48 | 8 | 5 | 1 |

The binding material was polycaprolactone.

The wells were then incubated (37° C., 5% CO₂) for half an hour to encourage adhesion to the material. 3 cm³ of fresh culture medium was then added to each well. Following further incubation for 24, 48 or 72 hours the pieces of flexible composite material were removed for examination.

Scanning electron microscopy (SEM) was performed on samples of the material to determine its structure prior to addition of the cells in both sterile and non-sterile form (see FIG. 1) and following incubation as described above to determine if cells adhered to the surface (see FIG. 2).

At the lower level of cell concentration (0.1-0.2×10⁶ cells/ml) there were few signs of the L929 fibroblasts attaching to the material after 24 hours. In the control wells the cells had proliferated and looked healthy and normal. After 48 hours, similar observations were obtained.

At the higher level of cell concentration (0.6-0.7×10⁶ cells/ml), cells looked normal in each well after 24 hours, with more cells being observed in the control well. Similar results were obtained at 48 hours.

The SEM results showed that although cells did adhere to the composite material, they did so at a low density. A far higher density of adhered cells was observed on the surface of the wells in which the experiment was carried out. In addition it was noted that the cells particularly did not adhere to regions of the fibres where polycaprolactone was present.

FIGS. 2a and 2b show SEM images (×100) of the non-sterile flexible composite material after 48 hours of incubation with L929 fibroblasts.

Thus this experiment demonstrates that the flexible biodegradable material, and in particular a composite material containing polycaprolactone, act in vitro to prevent cell adhesion.

1-27. (canceled)

28. A biodegradable material in the form of a flexible sheet for implantation in a human or non-human animal body, said biodegradable material comprising a composite of one or more coherent layers of a non-woven web of water soluble glass fibres together with a bio-compatible binder.

29. The biodegradable material as claimed in claim 28, wherein the bio-compatible binder is coated onto the surface of the glass fibres, or is in the form of a film fused to one or both sides of a layer of the glass fibres, or sandwiched between two or more layers of the glass fibres.

30. The biodegradable material as claimed in claim 28, wherein the binder is polycaprolactone, polyglycolic acid, polylactic acid, lactide/glycolide co-polymer or a mixture thereof.

31. The biodegradable material as claimed in claim 28, wherein the amount of binder in the composite material is 50% by weight or less.

32. The biodegradable material as claimed in claim 28, wherein the binder further comprises water soluble glass in powder form.

33. The biodegradable material as claimed in claim 28, wherein the binding material comprises a pharmacologically active agent.

34. The biodegradable material as claimed in claim 28, further comprising nutritional agents, growth factors, and healing agents, living cells, enzymes, chemical elements, charcoal, desloughing, debriding agents, or astringents.

35. The biodegradable material as claimed in claim 28, wherein the water soluble glass comprises one or more pharmacologically active agents releasable at a controllable rate.

36. The biodegradable material as claimed in claim 28, comprising a mixture of water soluble glass fibres of different formulations.

37. The biodegradable material as claimed in claim 28, comprising at least two biodegradable and/or non-biodegradable binders.

38. A method of treating an area of defective tissue in a patient, said method comprising using the biodegradable material as claimed in claim 28, to surround, cover or isolate said area of tissue.

39. The method as claimed in claim 38, wherein the material is attached to healthy or defective tissue by staples, sutures or biodegradable adhesive.

40. The method as claimed in claim 38, wherein the defective tissue is tendon, nerve, skin, bone, cardiovascular or abdominal tissue or dura matter.

41. The method as claimed in claim 38, comprising the step of positioning the biodegradable material between two internal tissue surfaces.

42. The method as claimed in claim 38, comprising the step of forming the biodegradable material into a tube around the area of tissue.

43. The method as claimed in claim 38, comprising the step of positioning the biodegradable material over an external area of tissue as a dressing.

44. The method as claimed in claim 43, wherein dermal cells are provided on the biodegradable material prior to application to the external area of tissue.

45. A method of reducing adhesion formation following surgery in a patient, said method comprising inserting the biodegradable material as claimed in claim 28 into the patient during surgery, and locating said material between the tissue surfaces where prevention of adhesion is required.

46. A method of repairing a damaged or severed nerve or tendon, said method comprising surrounding said damaged nerve or tendon, or the severed ends thereof with the biodegradable material of claim 28.

47. The biodegradable material as claimed in claim 28 for use in the treatment of an area of defective tissue, to protect said area of defective tissue, to promote healthy healing thereof or to prevent adhesion formation.

48. A surgical implant comprising the biodegradable material as claimed in claim 28.

49. A method of producing a biodegradable material in the form of a flexible sheet suitable for implantation into a patient's body, said method comprising:
a) forming one or more layers of water soluble glass fibres by:
i) winding glass fibres onto a drum;
ii) cutting along the length of the drum and removing the substantially aligned glass fibres therefrom; and
iii) pulling the glass fibers in a lateral direction to form a non-woven web.

50. The method as claimed in claim 49, wherein the fibres are formed into a coherent layer by at least one of the following steps:
i) fusing the fibres together by partial melting or dissolution; or
ii) needle-punching the layer of fibres to form a non-woven felt; or
iii) providing a binding material to adhere said layer to form a composite sheet.

51. The method as claimed in claim 49, wherein the binding material is adhered to the water soluble glass fibres by:
a) melting or dissolving the binding material in an appropriate solvent;
b) applying the binding material to the fibres by dipping, spraying or pouring to form a fibre/binder composite; and
c) cooling, curing or drying the fibre/binder composite.

52. The method as claimed in claim 49, wherein the binder is produced as a film and is then adhered to the water soluble glass layer by heat, solvent or adhesive.

53. A biodegradable material obtainable according to the method of claim 49.
